(19) United States
(12) Patent Application Publication
(10) Pub. No.: US 2002/0169404 A1
(43) Pub. Date: Nov. 14, 2002

(54) Bandage

(76) Inventor: Marsha T. Olderr, Hattiesburg, MS (US)

Correspondence Address: Stephen R. Greiner, Esquire, GREINER LAW OFFICES, P.C., Suite 110, 6701 Democracy Blvd., Bethesda, MD, 20817, US

(21) Appl. No.: 09/725,117

(22) Filed: Nov. 29, 2000

(51) Int. Cl. A61F 5/00

(52) U.S. Cl. 602/41; 602/63

(57) ABSTRACT

A bandage for covering and immobilizing a digit of a hand or foot. The bandage includes a cylindrical sleeve having an open inner end and a closed outer end defining an interior cavity. The sleeve has at least one pocket with an interior passage extending along the length of the sleeve and being positioned adjacent to the interior cavity. A splint is releasably held within the interior passage of the pocket. An absorbent lining for snugly encircling the digit of a user is positioned within the interior cavity and affixed to the sleeve.

### FIELD OF THE INVENTION

The present invention relates generally to skin laceration or wound covers and, more particularly, to such products having backing layers with a particular structural features.

### BACKGROUND OF THE INVENTION

A laceration or bruise on the tip of a finger is difficult to cover with a conventional, adhesive bandage. As is well known, the conventional bandage does not conform well to the compound curves found at a fingertip and tends to gather loosely when applied. Within a short period of time the bandage falls off leaving the wound uncovered. The problem repeats itself when such a bandage is replaced.

Thimble-like bandages that cover fingertip wounds have been proposed in the past. These bandages, generally speaking, have been complex and costly in construction. Furthermore, they have not done a particularly good job in immobilizing wounded digits, a significant shortcoming in situations where sprains or bone fractures are present. Thus, the known bandages for covering fingers have not seen great commercial success.

### SUMMARY OF THE INVENTION

In light of the problems associated with the known finger bandages, it is a principal object of the invention to provide a bandage of simplified construction that will cover the finger or toe of a user and protect a wound thereon. The bandage may be selectively reinforced to immobilize a wounded digit. The bandage may, therefore, be employed in a variety of situations medical situation.

It is another object of the invention to provide a bandage of the type described that can be used with minimal training and without resort to any additional tools.

It is an object of the invention to provide improved elements and arrangements thereof in a bandage for the purposes described which is lightweight in construction, inexpensive to manufacture, and dependable in use.

Briefly, the bandage in accordance with this invention achieves the intended objects by featuring a sleeve formed of a resilient material and having an open inner end and a closed outer end. The sleeve has a number of radially spaced pockets each with an interior passage extending along the length of the sleeve. A splint is positioned within the interior passage of each of the pockets. Each splint is a strip of rigid plastic material whose dimensions are substantially the same as that of the interior passage within which it is positioned. A gauze lining, for snugly encircling the digit of a user, is positioned within the sleeve and affixed thereto. The sleeve is provided with a number of perforations for ventilating its interior.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a perspective view of a bandage in accordance with the present invention.

FIG. 2 is a side view of the bandage of FIG. 1 with portions broken away to reveal details thereof.

FIG. 3 is a cross-sectional view taken along line 3-3 of FIG. 2.

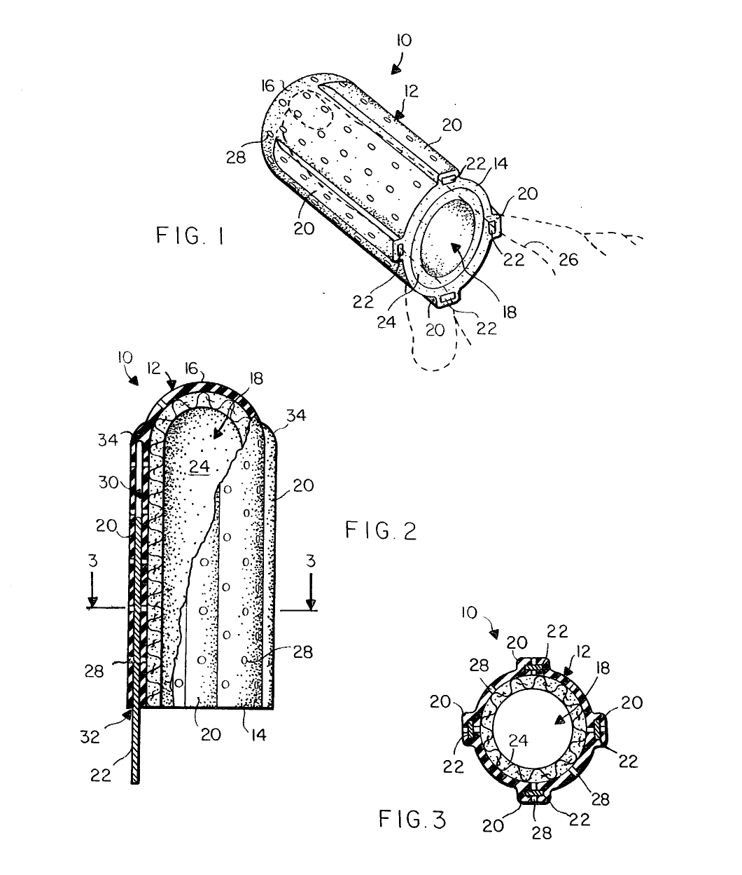

Similar reference characters denote corresponding features consistently throughout the accompanying drawings.

### DETAILED DESCRIPTION OF THE PREFERRED EMBODIMENT

Referring now to the FIGS., a bandage in accordance with the present invention is shown at 10. Bandage 10 includes a resilient sleeve 12 having a cylindrical configuration, with an open inner end 14 and a closed outer end 16, which defines an interior cavity 18. A number of pockets 20 are provided in sleeve 12 so as to releasably hold immobilizing splints 22 along the length of cavity 18. An absorbent lining 24 is positioned within cavity 18 and affixed to sleeve 12.

Sleeve 12 is formed from latex rubber or other suitable, plastic material. As shown, sleeve 12 is provided with a length sufficient to receive the digit of a user such as finger 26 and has a generally constant diameter from inner end 14 to outer end 16. Outer end 16 is hemispherical to conform to the shape of the tip of finger 26. Sleeve 12 is provided with perforations as at 28 along its length for ventilation.

Four pockets 20 are radially positioned around sleeve 12 and are oriented about ninety degrees apart. Each of the pockets 20 has an interior passage 30 extending along the length of sleeve 12 from an opening 32 at inner end 14 to a closed terminal end 34 adjacent outer end 16. Perforations 28 may penetrate pockets 20.

Splints 22 are snugly yet releasably positioned within pockets 20. Splints 22 preferably comprise strips of rigid plastic material whose dimensions closely mirror those of interior passages 30. The height of each splint 22 is preferably less than its width so as to maintain a low profile for sleeve 12 and a cavity 18 free of interior projections tending to press against finger 26.

Absorbent lining 24 is formed of sterile gauze or other absorbent material and extends over the entire interior surface of sleeve 12 from outer end 16 to inner end 14. Lining 24 is dimensioned to completely enclose finger 26 and covers the top, bottom, sides and tip of finger 26 to protect it and any wound thereon. Lining 24 may be formed from a web adhesively fastened as a single unit within sleeve 12 or applied as an unconsolidated material to the interior of sleeve 12 by other suitable processes.

Use of bandage 10 is straightforward. First, finger 26 having a cut, sprain, bruise, fracture or other wound and bearing a suitable dressing like a bactericide is inserted into the inner end 14 of sleeve 12 so that lining 24 fully engages and surrounds finger 26. Then, if finger 26 requires immobilization for proper healing, splints 22 are inserted into passages 30 of pockets 20 to stiffen sleeve 12 to the proper degree. Splints 22 may be omitted from some of the pockets 20 or partially inserted into others to obtain the desired amount of flexibility. Finally, if a dressing is used or if an open wound is present, bandage 10 is periodically removed from finger 26, discarded, and replaced with a fresh one. When the wound is healed, use of bandage 10 is discontinued.

While the invention has been described with a high degree of particularity, it will be appreciated by those skilled in the art that modifications may be made thereto. For example, the number and location of the pockets 20 and splints 22 can be varied as essentially any number could be employed in the invention. Also, sleeve 12 may be made in a variety of lengths and diameters to accommodate fingers, toes or other body parts of different sizes. Therefore, it is to be understood that the present invention is not limited to the sole embodiment described above, but encompasses any and all embodiments within the scope of the following claims.

I claim:

1. A bandage, comprising:
a cylindrical sleeve having an open inner end and a closed outer end defining an interior cavity, said sleeve having a pocket with an interior passage extending along the length of said sleeve and adjacent to said interior cavity;
a splint releasably held within said interior passage of said pocket; and,
an absorbent lining for snugly encircling the digit of a user, said absorbent lining positioned within said interior cavity and affixed to said sleeve.

2. The bandage according to claim 1 wherein said sleeve is provided with a plurality of perforations for ventilating said interior cavity.

3. A bandage, comprising:
a cylindrical sleeve formed of a resilient material and having an open inner end and a closed outer end defining an interior cavity, said sleeve having a plurality of pockets each with an interior passage extending along the length of said sleeve and being adjacent to said interior cavity;
a plurality of splints each being respectively positioned within said interior passage of one of said pockets; and,
a gauze lining for snugly encircling the digit of a user, said absorbent lining positioned within said interior cavity and affixed to said sleeve.

4. The bandage according to claim 4 wherein said sleeve is provided with a plurality of perforations for ventilating said interior cavity.

5. A bandage, comprising:
a cylindrical sleeve formed of a resilient material and having an open inner end and a closed outer end defining an interior cavity, said sleeve having a plurality of radially spaced pockets each with an interior passage extending along the length of said sleeve and being adjacent to said interior cavity, each of said pockets having an opening at said inner end of said sleeve and a closed terminal end adjacent said outer end of said sleeve;
a plurality of splints each being respectively positioned within said interior passage of one of said pockets, each of said splints being a strip of rigid plastic material whose dimensions are substantially the same as that of one said interior passage; and,
a gauze lining for snugly encircling the digit of a user, said absorbent lining positioned within said interior cavity and affixed to said sleeve.

6. The bandage according to claim 5 wherein said sleeve is provided with a plurality of perforations for ventilating said interior cavity.
